(19) United States
(12) Patent Application Publication
(10) Pub. No.: US 2025/0002999 A1
(43) Pub. Date: Jan. 2, 2025

(54) COVID BIOASSAY TO DETERMINE DISEASE SEVERITY AND THERAPY

(71) Applicant: VDS723, LLC, Scottsdale, AZ (US)

(72) Inventor: Divyen Patel, Schoharie, NY (US)

(21) Appl. No.: 18/635,961

(22) Filed: Apr. 15, 2024

Related U.S. Application Data
(60) Provisional application No. 63/460,021, filed on Apr. 17, 2023.

(51) Int. Cl. C12Q 1/6883; C12Q 1/6851

(52) U.S. Cl. CPC C12Q 1/6883; C12Q 1/6851; C12Q 2600/118; C12Q 2600/156

(57) ABSTRACT

Provided herein is an assay to identify patients more likely to progress to severe COVID upon COVID infection and methods to treat or prevent severe COVID using this assay.

### CROSS-REFERENCE TO RELATED APPLICATIONS

This application claims the benefit of and priority to U.S. Provisional Application No. 63/460,021, filed Apr. 17, 2023, the contents of which are incorporated by reference in its entirety.

### BACKGROUND

Discovered in Wuhan China in late 2019, Severe Acute Respiratory Syndrome coronavirus 2 (SARS-CoV-2) and the coronavirus disease 2019 (Covid-19) rapidly evolved into a global pandemic. Since its first discovery (Na Zhu, 2020), 378 million cases have been reported worldwide and 5.67 million deaths attributed to the Coronavirus disease. While the majority of patients suffer mild to moderate symptoms, or display no symptoms at all, the mortality rate of the Coronavirus disease is predominantly driven by a subgroup of patients that display severe respiratory failure related to interstitial pneumonia in both lungs and severe respiratory distress syndrome (David A Berlin, 2020).

The pathogenesis of this subgroup of patients is poorly understood, but there is evidence of a pathophysiology distinct from that of other patients with the Coronavirus disease. For example, corticosteroids have proven beneficial in treating critical respiratory failure in Coronavirus patients, but are harmful in treating patients with the Coronavirus disease that do not display respiratory failure (Inga Patarčić, 2015). Thus, a need exists in the art to identify patients who are more likely to experience severe Covid infections so that preventive and personalized therapies can be administered to those in need. This disclosure satisfies this need for preventive and provides related advantages as well.

### SUMMARY OF THE DISCLOSURE

In nature, organisms of the same species usually differ from each other in some aspects, e.g., their appearance. The differences are genetically determined and are referred to as polymorphism. Genetic polymorphism is the occurrence in a population of two or more genetically determined alternative phenotypes due to different alleles. Polymorphism can be observed at the level of the whole individual (phenotype), in variant forms of proteins and blood group substances (biochemical polymorphism), morphological features of chromosomes (chromosomal polymorphism) or at the level of DNA in differences of nucleotides (DNA polymorphism).

With a view to delineating host genetic factors that contributed to severe respiratory failure in Coronavirus patients and progression to severe disease, genome wide association studies (GWAS) were carried out on cohorts of patients.

The following 4 SNPS were individually and independently validated as being predictive of disease severity and likelihood of disease progression. While the presence of one SNP is predictive, two or, three or, or all four raises the likelihood of progression to severe disease and the severity of that disease as the number of SNPs are present in the sample.

Table 1 Shows the rsIDs for the SNPs and the Corresponding Mutation from Wildtype and the Phenotypic Impact (Clinical Impact).

| rsID SNP Containing | | |
|---|---|---|
| Polymorphisms of | Polymorphism | |
| Signficance | WT to SNP | Clinical Risk |
| rs111837807 | T to A | Disease severity |
| rs117169628 | G to A | Disease severity |
| rs1405655 | T to C | Disease severity |
| rs2236757 | G to A | Disease severity |

While probes and primers for the detection of these SNPs are known in the art, Applicant has discovered that the exemplified probe/primer pairs provided in Appendix A unexpected higher specificity and predictability as compared to prior art probes and primers (data not shown).

Thus, provided herein is a method for identifying whether a patient is at risk for progressing to severe COVID comprising, or consisting essentially of, or yet further consisting of screening a biological sample isolated from the patient for 1 or more polymorphisms selected from the group shown in Table 1, and identifying that the patient is at risk for progressing to severe COVID if 1 or more polymorphisms are identified in the sample.

Also provided is a method for identifying whether a patient is not at risk for progressing to severe COVID comprising, or consisting essentially of, or yet further consisting of screening a biological sample isolated from the patient for 1 or more polymorphisms selected from the group shown in Table 1, and identifying that the patient is not at risk for progressing to severe COVID if none of the polymorphisms are identified in the sample.

Further provided is a method for treating a patient likely to progress to severe COVID infection comprising, or consisting essentially of, or yet further consisting of administering an effective amount of a therapy selected from a COVID vaccine, a COVID booster, a corticosteroid, Paxlovid, Remdesivir, or Molnupiravir, or other equivalent therapy recommended for patients who are at high risk for progressing to severe COVID, wherein the patient has been identified for the treatment by screening a biological sample isolated from the patient for 1 or more polymorphisms selected from the group of Table 1, and identifying that the patient is at risk for progressing to severe COVID if 1 or more polymorphisms are identified in the sample.

Also provided is a method for selecting a patient for a therapy to treat COVID comprising, or consisting essentially of, or yet further consisting of screening a biological sample isolated from the patient for 1 or more polymorphisms selected from the group of Table 1, and selecting a therapy to treat a patient at risk for progressing to severe COVID if 1 or more polymorphisms are identified in the sample.

Further provide is a probe and primer pair as shown in Appendix A and a preservative and/or a stabilizer for the probes and/or primer pairs.

This disclosure also provides a kit for performing the method of any preceding claim, comprising reagents to identify or determine the genotype of the sample and instructions for use.

### DETAILED DESCRIPTION OF THE DISCLOSURE

Throughout this disclosure, various publications, patents and published patent specifications are referenced by an identifying citation. The disclosures of these publications, patents and published patent specifications are hereby incorporated by reference into the present disclosure to more fully describe the state of the art to which this disclosure pertains.

As used herein, certain terms may have the following defined meanings. As used in the specification and claims, the singular form “a,” “an,” and “the” include singular and plural references unless the context clearly dictates otherwise. For example, the term “a cell” includes a single cell as well as a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the composition or method. “Consisting of” shall mean excluding more than trace elements of other ingredients for claimed compositions and substantial method steps. Embodiments defined by each of these transition terms are within the scope of this disclosure. Accordingly, it is intended that the methods and compositions can include additional steps and components (comprising) or alternatively including steps and compositions of no significance (consisting essentially of) or alternatively, intending only the stated method steps or compositions (consisting of).

All numerical designations, e.g., pH, temperature, time, concentration, and molecular weight, including ranges, are approximations which are varied (+) or (−) by increments of 0.1.

It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

In one aspect, the term “equivalent” or “biological equivalent” of an antibody means the ability of the antibody to selectively bind its epitope protein or fragment thereof as measured by ELISA or other suitable methods. Biologically equivalent antibodies include, but are not limited to, those antibodies, peptides, antibody fragments, antibody variant, antibody derivative and antibody mimetics that bind to the same epitope as the reference antibody.

In one aspect, the term “equivalent” of “chemical equivalent” of a chemical means the ability of the chemical to selectively interact with its target protein, DNA, RNA or fragment thereof as measured by the inactivation of the target protein, incorporation of the chemical into the DNA or RNA or other suitable methods. Chemical equivalents include, but are not limited to, those agents with the same or similar biological activity and include, without limitation a pharmaceutically acceptable salt or mixtures thereof that interact with and/or inactivate the same target protein, DNA, or RNA as the reference chemical.

The term “allele,” which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions and insertions of nucleotides. An allele of a gene can also be a form of a gene containing a mutation.

The term “genotype” refers to the specific allelic composition of an entire cell or a certain gene and in some aspects a specific polymorphism associated with that gene, whereas the term “phenotype” refers to the detectable outward manifestations of a specific genotype.

As used herein, the term “determining the genotype of a cell or tissue sample” intends to identify the genotypes of polymorphic loci of interest in the cell or tissue sample. In one aspect, a polymorphic locus is a single nucleotide polymorphic (SNP) locus. If the allelic composition of a SNP locus is heterozygous, the genotype of the SNP locus can be identified as “X/Y” wherein X and Y are two different nucleotides. If the allelic composition of a SNP locus is heterozygous, the genotype of the SNP locus can be identified as “X/X” wherein X identifies the nucleotide that is present at both alleles.

The term “genetic marker” refers to an allelic variant of a polymorphic region of a gene of interest and/or the expression level of a gene of interest.

The term “polymorphism” refers to the coexistence of more than one form of a gene or portion thereof. A portion of a gene of which there are at least two different forms, i.e., two different nucleotide sequences, is referred to as a “polymorphic region of a gene.” A polymorphic region can be a single nucleotide, the identity of which differs in different alleles.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “isolated” as used herein refers to molecules or biological or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

In one embodiment, the term “disease” or “disorder” as used herein refers to a coronavirus infection, a status of being diagnosed with such infection, a status of being suspect of having such infection, a status of having being exposed to a coronavirus, or a status of at high risk of being exposed to a coronavirus. In one embodiment, the coronavirus is a respiratory virus. In a further embodiment, the disease is Coronavirus disease 2019 (COVID-19) caused by SARS-CoV-2. In yet a further embodiment, the disease is Severe acute respiratory syndrome (SARS) caused by SARS-CoV-1.

Coronaviruses constitute the subfamily Orthocoronavirinae, in the family Coronaviridae, order Nidovirales, and realm Riboviria. They are enveloped viruses with a positive-sense single-stranded RNA genome and a nucleocapsid of helical symmetry. The genome size of coronaviruses ranges from approximately 26 to 32 kilobases, one of the largest among RNA viruses. They have characteristic club-shaped spikes that project from their surface, which in electron micrographs create an image reminiscent of the solar corona, from which their name derives.

In some embodiments, the coronavirus as used herein refers to a severe acute respiratory syndrome (SARS) associated coronavirus (SARS-CoV). In some embodiments, the coronavirus is either or both of SARS-CoV-1 and SARS-CoV-2. In some embodiments, the coronavirus comprises a virus selected from the group consisting of an Alphacoronavirus; a Colacovirus such as Bat coronavirus CDPHE15; a Decacovirus such as Bat coronavirus HKU10 or Rhinolophus ferrumequinum alphacoronavirus HuB-2013; a Duvinacovirus such as Human coronavirus 229E; a Luchacovirus such as Lucheng Rn rat coronavirus; a Minacovirus such as a Ferret coronavirus or Mink coronavirus 1; a Minunacovirus such as Miniopterus bat coronavirus 1 or Miniopterus bat coronavirus HKU8; a Myotacovirus such as Myotis ricketti alphacoronavirus Sax-2011; a nyctacovirus such as Nyctalus velutinus alphacoronavirus SC-2013; a Pedacovirus such as Porcine epidemic diarrhea virus or Scotophilus bat coronavirus 512; a Rhinacovirus such as Rhinolophus bat coronavirus HKU2; a Setracovirus such as Human coronavirus NL63 or NL63-related bat coronavirus strain BtKYNL63-9b; a Tegacovirus such as Alphacoronavirus 1; a Betacoronavirus; a Embecovirus such as Betacoronavirus 1, Human coronavirus OC43, China Rattus coronavirus HKU24, Human coronavirus HKU1 or Murine coronavirus; a Hibecovirus such as Bat Hp-betacoronavirus Zhejiang2013; a Merbecovirus such as Hedgehog coronavirus 1, Middle East respiratory syndrome-related coronavirus (MERS-CoV), Pipistrellus bat coronavirus HKU5 or Tylonycteris bat coronavirus HKU4; a Nobecovirus such as Rousettus bat coronavirus GCCDC1 or Rousettus bat coronavirus HKU9, a Sarbecovirus such as a Severe acute respiratory syndrome-related coronavirus, Severe acute respiratory syndrome coronavirus (SARS-CoV) or Severe acute respiratory syndrome coronavirus 2 (SARS-CoV-2, COVID-19); a Deltacoronavirus; an Andecovirus such as Wigeon coronavirus HKU20; a Buldecovirus such as Bulbul coronavirus HKU11, Porcine coronavirus HKU15, Munia coronavirus HKU13 or White-eye coronavirus HKU16; a Herdecovirus such as Night heron coronavirus HKU19; a Moordecovirus such as Common moorhen coronavirus HKU21; a Gammacoronavirus; a Cegacovirus such as Beluga whale coronavirus SW1; and an Igacovirus such as Avian coronavirus.

Symptoms of a coronavirus infection include, but are not limited to, mild symptoms, such as fatigues, tingling, tingling or numbness in the hands and feet, dizziness, confusion, brain fog, body ache, chills, loss of appetite, nausea, vomiting, abdominal pain or discomfort, loss of smell, inability to taste, muscle weakness, photophobia, adenopathy, headaches, cough, dry cough, shortness of breath, sore throat, lower extremity weakness/numbness, diarrhea, low blood O₂, sneezing, runny nose or post-nasal drip; severe symptoms, such as ventilatory use, high fever, severe cough, delirium, seizures, stroke, systematic inflammation, cytokine storm; and other symptoms, such as fever, swollen adenoids, pneumonia, bronchitis, and Dyspnea.

The National Institute of Health (NIH) indicates that patients with SARS-CoV-2 infection can experience a range of clinical manifestations, from no symptoms to critical illness. In general, adults with SARS-CoV-2 infection can be grouped into the following severity of illness categories; however, the criteria for each category may overlap or vary across clinical guidelines and clinical trials, and a patient's clinical status may change over time. Asymptomatic or presymptomatic infection: Individuals who test positive for SARS-CoV-2 using a virologic test (i.e., a nucleic acid amplification test [NAAT] or an antigen test) but have no symptoms consistent with COVID-19. Mild illness: Individuals who have any of the various signs and symptoms of COVID-19 (e.g., fever, cough, sore throat, malaise, headache, muscle pain, nausea, vomiting, diarrhea, loss of taste and smell) but do not have shortness of breath, dyspnea, or abnormal chest imaging. Moderate illness: Individuals who show evidence of lower respiratory disease during clinical assessment or imaging and who have an oxygen saturation measured by pulse oximetry (SpO₂)≥94% on room air at sea level. Severe illness: Individuals who have SpO₂<94% on room air at sea level, a ratio of arterial partial pressure of oxygen to fraction of inspired oxygen (PaO₂/FiO₂)<300 mm Hg, a respiratory rate >30 breaths/min, or lung infiltrates >50%. Critical illness: Individuals who have respiratory failure, septic shock, and/or multiple organ dysfunction. Reproduced from https://www.covid19treatmentguidelines.nih.gov/overview/clinical-spectrum/, last updated Mar. 6, 2023.

SpO₂ is a key parameter for defining the illness categories listed above. However, pulse oximetry has important limitations. Clinicians who use SpO₂ when assessing a patient must be aware of those limitations and conduct the assessment in the context of that patient's clinical status. Reproduced from https://www.covid19treatmentguidelines.nih.gov/overview/clinical-spectrum/, last updated Mar. 6, 2023.

Patients aged ≥50 years are at a higher risk of progressing to severe COVID-19. Other underlying conditions associated with a higher risk of severe COVID-19 include asthma, cancer, cardiovascular disease, chronic kidney disease, chronic liver disease, chronic lung disease, diabetes, advanced or untreated HIV infection, obesity, pregnancy, cigarette smoking, and being a recipient of immunosuppressive therapy or a transplant. Health care providers should closely monitor patients with these conditions until they achieve clinical recovery. Reproduced from https://www.covid19treatmentguidelines.nih.gov/overview/clinical-spectrum/, last updated Mar. 6, 2023.

The initial evaluation for patients may include chest imaging (e.g., X-ray, ultrasound or computed tomography scan) and an electrocardiogram. Laboratory testing should include a complete blood count with differential and a metabolic profile, including liver and renal function tests. Although inflammatory markers such as C-reactive protein (CRP), D-dimer, and ferritin are not routinely measured as part of standard care, results from such measurements may have prognostic value. Reproduced from:

https://www.covid19treatmentguidelines.nih.gov/overview/clinical-spectrum/, last updated Mar. 6, 2023.

The definitions for the severity of illness categories also apply to pregnant patients. However, the threshold for certain interventions is different for pregnant and nonpregnant patients. For example, oxygen supplementation for pregnant patients is generally used when SpO₂ falls below 95% on room air at sea level to accommodate the physiologic changes in oxygen demand during pregnancy and to ensure adequate oxygen delivery to the fetus. Reproduced from https://www.covid19treatmentguidelines.nih.gov/overview/clinical-spectrum/, last updated Mar. 6, 2023.

If laboratory parameters are used for monitoring pregnant patients and making decisions about interventions, clinicians should be aware that normal physiologic changes during pregnancy can alter several laboratory values. In general, leukocyte cell count increases throughout gestation and delivery and peaks during the immediate postpartum period. This increase is mainly due to neutrophilia. D-dimer and CRP levels also increase during pregnancy and are often higher in pregnant patients than in nonpregnant patients. Reproduced from https://www.covid19treatmentguidelines.nih.gov/overview/clinical-spectrum/, last updated Mar. 6, 2023.

In children with COVID-19, radiographic abnormalities are common and, for the most part, should not be the only criteria used to determine the severity of illness. The normal values for respiratory rate also vary with age in children, therefore, hypoxemia should be the primary criterion used to define severe COVID-19, especially in younger children. In a small subset of children and young adults, SARS-CoV-2 infection may be followed by the severe inflammatory condition multisystem inflammatory syndrome in children (MIS-C). Reproduced from https://www.covid19treatmentguidelines.nih.gov/overview/clinical-spectrum/, last updated Mar. 6, 2023.

As used herein, an inflammation refers to a biological response of a subject to harmful stimuli, such as pathogens (for example a virus), damaged cells, irritants, and involves immune cells, blood vessels and molecular mediators. On the other hand, infection describes the interaction between the action of microbial (such as a viral) invasion and the reaction of the subject's inflammatory response—the two components are considered together when discussing an infection, and the word is used to imply a microbial invasive cause for the observed inflammatory reaction. The five classical signs of inflammation are heat, pain, redness, swelling, and loss of function. In one embodiment, a c-reactive protein test measures the level of c-reactive protein (CRP) in the blood thus measuring inflammation in a subject. In another embodiment, erythrocyte sedimentation rate (ESR), and/or plasma viscosity (PV) detect increase in protein in the blood, and used as markers of inflammation. Other methods can be used to qualify or quantify an inflammation, such as via quantifying amount(s) of one or more of cell-derived mediators, such as a cytokine, an interleukin, a chemokine, or others (including but not limited to lysosome granules, histamine, leukotriene B4, LTC4, LTD4, 5-oxo-eicosatetraenoic acid, 5-HETE, Prostaglandin, Nitric oxide or Tryptase). In some embodiments, the inflammation comprises an elevated cytokine, optionally selected from the group consisting of interferons (IFN) such as Type I IFNs (IFN-α and IFN-β), type II IFN (IFN-γ), Lambda IFNs (IFN-λ1, IFN-λ2, and IFN-λ3); interleukins such as IL-1α and IL-1β; chemokines such as CXCL8 (IL-8), CCL2 (monocyte chemoattractant protein 1 [MCP-1]), and CCL11 (eotaxin); colony-stimulating factors (CSFs), such as granulocyte-macrophage colony-stimulating factor (GM-CSF), macrophage colony-stimulating factor (M-CSF), and granulocyte colony-stimulating factor (G-CSF); tumor necrosis factor (TNF); and combinations thereof. The elevated level may be determined using any medically recognized technique, for example, mass spectrometry, enzyme-linked immunosorbent assay (ELISA), or immunohistochemistry. In one embodiment, the inflammation comprises a systematic cytokine storm.

Prior art tests are available to detect the virus after infection using for example polymerase chain reaction (PCR) or immunoassay may be used in combination with the disclosed test and treatments. In some embodiments, a method as disclosed herein further comprises detecting a coronavirus via a test known in the art. In one embodiment, active viral infection refers to an ongoing infection wherein the virus is making copies of itself and producing new virus. Such active viral infection may be detected via polymerase chain reaction (PCR). Non-limiting examples of primers and probes suitable for use in the PCR include 2019-nCoV CDC Probe and Primer Kit for SARS-CoV-2 (BioSearch Technologies, Catalog No. KIT-nCoV-PP1-1000), 2019-nCoV Kit, 500 rxn (Integrated DNA Technologies (IDT), Catalog No. 10006606) and 2019-nCoV Kit, 1000 rxn (Integrated DNA Technologies (IDT), Catalog No. 10006770). Also see, www.cdc.gov/coronavirus/2019-ncov/lab/rt-pcr-panel-primer-probes.html and www.cdc.gov/coronavirus/2019-ncov/downloads/List-of-Acceptable-Commercial-Primers-Probes.pdf. Suitable protocols for performing such tests can be found at www.cdc.gov/coronavirus/2019-ncov/lab/virus-requests.html, www.fda.gov/media/134922/download, www.cdc.gov/coronavirus/2019-ncov/downloads/processing-sputum-specimens.pdf, www.fda.gov/media/134922/download, www.fda.gov/media/134919/download, and www.fda.gov/media/134922/download. In some embodiments, diagnostic assays for COVID-19 based on detecting antibodies may be combined with the disclosure herein, such as those discussed by Lisboa Bastos M et al. (Diagnostic accuracy of serological tests for covid-19: systematic review and meta-analysis. BMJ. 2020 Jul. 1; 370: m2516. doi: 10.1136/bmj.m2516).

Commercially available tests include, but not limited to those listed in Table 2 below.

TABLE 2
Commercially available tests for SARS-CoV-2 and COVID-19

| Company Name | Test Name |
|---|---|
| 3D Medicines | SARS-CoV-2 and Influenza A & B RT-qPCR Detection |
| | Kit |
| Abbott | SARS-CoV-2 IgG test |
| Abbott | ID Now COVID-19 |
| Abbott | Abbott RealTime SARS-CoV-2 EUA test |
| Anatolia Geneworks | Bosphore Novel Coronavirus (2019-nCoV) Detection Kit |
| ARUP Laboratories | COVID-19 |
| A*STAR, Tan Tock Seng Hospital | A*STAR Fortitude 2.0 |
| of Singapore | |
| Assure Tech | COVID-19 IgG/IgM Rapid Test Device |
| Atila BioSystems | iAMP COVID-19 Detection Kit |
| AusDiagnostics | AusDiagnostics SARS-CoV-2, influenza, RSV panel |
| Autobio Diagnostics | Anti-SARS-CoV-2 Rapid Test |
| Avellino Lab | Avellino SARS-CoV-2/COVID-19 (AvellinoCoV2) |
| Bako Diagnostics | BakoDx SARS-CoV-2 RNA test |
| Baptist Hospital Miami | COVID-19 RT-PCR Test |
| Pathology/Laboratory Medicine | |
| Lab | |
| Becton Dickinson | BD SARS-CoV-2 Reagents for BD MAX System |
| Becton Dickinson, BioGx | BioGX SARS-CoV-2 Reagents for the BD MAX System |
| Beijing Decombio Biotechnology | Novel Coronavirus IgM/IgG Combo Rapid Test-Cassette |
| Beijing Diagreat Biotechnologies | 2019-nCoV IgG, IgM Antibody Determination Kits |
| | 2019-nCoV IgG/IgM Antibody Rapid Test Kit |
| Beijing Kewei Clinical Diagnostic | Genonto RapidTest10 COVID-19 IgG/IgM Antibody |
| Reagent | Rapid Test Kit |
| Beijing O&D Biotech | Coronavirus disease (COVID-19) Total Antibody Rapid |
| | Test (Colloidal Gold) |
| Beroni Group | SARS-CoV-2 IgG/IgM Antibody Detection Kit |
| BGI | Real-Time Fluorescent RT-PCR kit for detecting SARS- |
| | 2019-nCoV |
| Biodesix | SARS-CoV-2 Droplet Digital PCR (ddPCR) test |
| Biolidics | 2019-nCoV IgG/IgM Detection Kit (Colloidal Gold) |
| BioMedomics | COVID-19 IgM-IgG Rapid Test |
| BioMérieux | SARS-COV-2 R-GENE test |
| BioMérieux/BioFire Defense | BioFire COVID-19 test |
| Bioneer | AccuPower COVID-19 Real-Time RT-PCR Kit, |
| | AccuPower SARS-CoV-2 Real-Time RT-PCR Kit |
| Bio-Rad Laboratories | SARS-CoV-2 Total Ab test |
| BioReference Laboratories | Novel Coronavirus COVID-19 |
| Boston Children's Hospital | Childrens-Altona-SARS-CoV-2 assay |
| Infectious Diseases Diagnostic | |
| Laboratory (IDDL) | |
| BTNX | Rapid Response COVID-19 IgG/IgM Test Cassette |
| Cellex | qSARS-CoV-2 IgG/IgM Rapid Test |
| Centers for Disease Control and | CDC 2019-Novel Coronavirus (2019-nCoV) Real-Time |
| Prevention (performed at qualified | RT-PCR Diagnostic Panel (CDC) |
| high-complexity CLIA | |
| laboratories designated by CDC) | |
| Cepheid | Xpert Xpress SARS-CoV-2 test |
| CerTest BioTec | ViaSure SARS-CoV-2 Real Time PCR Detection Kit |
| Chembio Diagnostics | DDP COVID-19 IgM/IgG System |
| Children's Hospital of Philadelphia | SARS-CoV-2 RT-PCR test |
| Infectious Disease Diagnostics | |
| Laboratory | |
| ChromaCode | HDPCR SARS-CoV-2 real-time PCR assay |
| CirrusDx Laboratories | CirrusDx SARS-CoV-2 Assay |
| Co-Diagnostics | Logix Smart Coronavirus Disease 2019 (COVID-19) Kit |
| Core Technology | CoreTest COVID-19 IgM/IgG Ab Test |
| Credo Diagnostics Biomedical | VitaPCR SARS-CoV2 Assay |
| DiaCarta | QuantiVirus SARS-CoV-2 test kit |
| Diagnostic Solutions Laboratory | COVID-19 Assay |
| DiaSorin Molecular | Simplexa COVID-19 Direct |
| Diatherix Eurofins | COVID-19 Panel |
| Diazyme Laboratories | Diazyme DZ-LITE SARS-CoV-2 IgG, IgM CLIA Kits |
| Eachy Biopharmaceuticals | AccuRapid SARS-CoV-2 IgM/IgG Test Kit (Lateral |
| | Flow Immunoassay) |
| Euroimmun/PerkinElmer | EuroRealTime SARS-CoV-2 |
| Euroimmun/PerkinElmer | Anti-SARS-CoV-2 ELISAs (IgA and IgG) |
| Exact Sciences | SARS-CoV-2 Test |
| Fosun Pharma USA | Fosun COVID-19 RT-PCR Detection Kit |
| Fulgent Genetics/MedScan | COVID-19 |
| Laboratory | |
| Genetic Signatures | EasyScreen SARS-CoV-2 detection kit |
| Genetron | Detection Kit for Novel Coronavirus (SARS-CoV-2) |
| | RNA (PCR-Fluorescence Probing) |
| GenMark Diagnostics | ePlex SARS-CoV-2 Test |
| Genomica/PharmMar Group | 2 kits: qCOVID-19, CLART COVID-19 |
| GenoSensor | GS COVID-19 RT-PCR Kit |
| Gnomegen | Gnomegen COVID-19 RT-Digital PCR Detection Kit |
| Gold Standard Diagnostics | SARS-CoV-2 IgG, IgM, IgA assays |
| Guangzhou Wondfo Biotech | SARS-CoV-2 Antibody Test |
| Hackensack University Medical | CDI Enhanced COVID-19 Test |
| Center (HUMC) Molecular | |
| Pathology Laboratory | |
| Hangzhou AllTest Biotech | AllTest 2019-nCoV IgG/IgM Rapid Test Cassette, |
| | AllTest COVID IgG/IgM Rapid Test Dipstick |
| Hangzhou Biotest Biotech | COVID-19 IgG/IgM Rapid Test Cassette |
| Hangzhou Clongene Biotech | Clungene COVID-19 IgM/IgG Rapid Test Cassette |
| Hangzhou Testsealabs | One Step SARS-CoV2 (COVID-19) IgG/IgM Test |
| Biotechnology | |
| Healgen Scientific | COVID-19 IgG/IgM Rapid Test Cassette(Whole |
| | Blood/Serum/Plasma) |
| Hologic | Panther Fusion SARS-CoV-2 assay |
| InBios International | Smart Detect SARS-CoV-2 rRT-PCR Kit |
| Innovita (Tangshan) Biological | 2019-nCoV Ab Test (Colloidal Gold) |
| Technology | |
| Integrated DNA | IDT 2019-novel coronavirus kit |
| Technologies/Danaher | |
| Integrity Laboratories | SARS-CoV-2 Assay |
| Ipsum Diagnostics | COV-19 IDx assay |
| Jiangsu Macro & Micro-Test Med- | SARS-CoV-2 IgM/IgG Rapid Assay Kit (Colloidal |
| Tech | Gold) |
| JN Medsys | ProTect Covid-19 kit |
| Kogene Biotech | 2019 Novel Coronavirus Real-time PCR Kit |
| KorvaLabs | Curative-Korva SARS-Cov-2 Assay |
| Laboratory Corporation of | LabCorp 2019 Novel Coronavirus test |
| America | |
| LGC, Biosearch Technologies | 2019-nCoV CDC-qualified Probe and Primer Kits for |
| | SARS-CoV-2 |
| Lifeassay Diagnostics | Test-it COVID-19 IgM/IgG Lateral Flow Assay |
| Luminex | ARIES SARS-CoV-2 Assay |
| Luminex | NxTAGCoV Extended Panel Assay |
| Maccura Biotechnology | SARS-CoV-2 Fluorescent PCR Kit |
| Massachusetts General Hospital | MGH COVID-19 qPCR assay |
| Medical Systems Biotechnology | Coronavirus Disease 2019 Antibody (IgM/IgG) |
| | Combined Test Kit |
| Mesa Biotech | Accula SARS-CoV-2 test |
| Mount Sinai Labs | COVID-19 ELISA IgG Antibody Test |
| Nanjing Liming Bio-products | SARS-CoV-2 IgM/IgG Antibody Rapid Test Kit |
| NanoResearch | NanoMedicina SARS-CoV-2 IgM/IgG Antibody Rapid |
| | Test |
| Nantong Diagnos Biotechnology | (2019-nCoV) New coronavirus Antibody Test (Colloidal |
| | Gold) |
| NeuMoDx Molecular | NeuMoDx SARS-CoV-2 Assay |
| Nirmidas Biotech | COVID-19 (SARS-CoV-2) IgM/IgG Antibody Detection |
| | Kit |
| Northwestern Medicine Diagnostic | SARS-Cov-2 Assay |
| Molecular Laboratory | |
| Novacyt/Primerdesign | COVID-19 Genesig Real-Time PCR assay |
| NY State Department of Health | New York SARS-CoV-2 Real-time Reverse |
| (performed at Wadsworth Center | Transcriptase (RT)-PCR Diagnostic Panel |
| and New York City Department of | |
| Health and Mental Hygiene, | |
| Public Health Laboratories) | |
| Orig3n | Orig3n 2019 Novel Coronavirus (COVID-19) Test |
| Ortho Clinical Diagnostics | Vitros Immunodiagnostic Products Anti-SARS-CoV-2 |
| | Total Reagent Pack and Calibrators |
| Osang Healthcare | GeneFinder COVID-19 Plus RealAmp Kit |
| PathoFinder | RealAccurate Quadruplex Corona-plus PCR Kit |
| PCL | COVID19 IgG/IgM Rapid Gold |
| PerkinElmer | PerkinElmer New Coronavirus Nucleic Acid Detection |
| | Kit |
| Phamatech | COVID19 IgG/IgM Rapid Test |
| Promedical | COVID-19 Rapid Test, Wondfo SARS-CoV-2 Antibody |
| | Test (Lateral Flow Method) |
| Qiagen | QiaStat-Dx Respiratory SARS-CoV-2 Panel |
| Quest Diagnostics | Coronavirus Disease 2019 (COVID-19) Test |
| Quidel | Lyra SARS-CoV-2 Assay |
| Rendu Biotechnology | 2019-nCoV detection kit |
| Roche | Cobas SARS-CoV-2 Test |
| Rutgers University Clinical | ThermoFisher - Applied Biosystems TaqPath COVID-19 |
| Genomics Laboratory | Combo Kit |
| ScienCell Research Laboratories | ScienCell SARS-CoV-2 Coronavirus Real-time RT-PCR |
| | (RT-qPCR) Detection Kit |
| SD Biosensor | Standard Q COVID-19 IgM/IgG Duo |
| Seegene | Allplex 2019-nCoV Assay |
| Sentinel Diagnostics | STAT-NAT COVID-19 HK kit, B kit |
| Shanghai Fosun Long March | novel coronavirus nucleic acid detection kit |
| Medical Science/Shanghai Fosun | |
| Pharmaceutical | |
| Shenzhen Landwind Medical | COVID-19 IgG/IgM Rapid Test Device |
| Snibe Diagnostics | Maglumi 2019-nCoV (SARS-CoV-2) IgM/IgG kits |
| SolGent | DiaPlexQ Novel Coronavirus (2019-nCoV) Detection kit |
| Specialty Diagnostic (SDI) | SDI SARS-CoV-2 Assay |
| Laboratories | |
| Stanford Health Care Clinical | SARS-CoV-2 PCR Assay |
| Virology Laboratory | |
| SureScreen Diagnostics | SureScreen COVID19 IgM/IgG Rapid Test Cassette |
| Suzhou Kangheshun Medical | SARS-CoV-2 IgG/IgM Rapid Test Cassette |
| Technology | |
| Systaaq Diagnostic Products | 2019-Novel Coronavirus (COVID-19) Real Time PCR |
| | Kit |
| Telepoint Medical Services | SARS-CoV-2 IgG/IgM Rapid Qualitative Test |
| Thermo Fisher Scientific | TaqPath COVID-19 Combo Kit, RT-PCR CE-IVD Kit |
| Tianjin Beroni Biotechnology | SARS-CoV-2 IgG/IgM Antibody Detection Kit |
| TIB Molbiol Syntheselabor | Sarbecovirus E-gene |
| Trax Management Services | Phoenix Dx 2019-CoV |
| United Biomedical | UBI SARS-CoV-2 ELISA |
| University of North Carolina | UNC Health SARS-CoV-2 real-time RT-PCR test |
| Medical Center | |
| Vela Diagnostics | ViroKey SARS-CoV-2 RT-PCR Test |
| Viracor Eurofins | Viracor SARS-CoV-2 assay |
| Vision Medicals | SARS-CoV-2 Clinical Sequencing assay |
| VivaChek Biotech (Hangzhou) | VivaDiag COVID-19 IgM/IgG Rapid Test |
| Yale New Haven Hospital Clinical | SARS-CoV-2 PCR test |
| Virology Laboratory | |
| YD Diagnostics | MolecuTech Real-Time COVID-19 |
| Zhejiang Orient Gene Biotech | COVID-19 IgG/IgM Rapid Test Cassette |
| Zhengzhou Fortune Bioscience | IgG/IgM Antibody Rapid Test Kits (Colloidal Gold |
| | Immunochromatography method) |
| Zhongshan Bio-Tech | SARS-CoV-2 IgM/IgG (GICA) |
| Zhuhai Encode Medical | Novel Coronavirus (COVID-19) IgG/IgM Rapid Test |
| Engineering | Device |
| Zhuhai Livzon Diagnostics | Diagnostic Kit for IgM/IgG Antibody to Coronavirus |
| | (SARS-CoV-2) (Colloidal Gold) |

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis.

As used herein, the term “recovery” refers to return to a normal and healthy state of health, mind, or strength prior to a disease as disclosed herein, or to a normal and healthy state of health, mind, or strength of a subject free of such disease. In some embodiments, a poor recovery refers to not substantially returning to or not returning to the normal and healthy state, or returning to the normal and healthy state over a long period of time, or death.

“An effective amount” or “therapeutically effect amount” intends to indicate the amount of a compound or agent administered or delivered to the patient which is most likely to result in the desired response to treatment.

As used herein, vaccine refers to a substance, such as a peptide, used to stimulate an immune response, such as production of antibodies, and provide immunity against one or several diseases. Vaccination or a grammatical variation thereof refers to administration of a vaccine to a subject to help the immune system develop protection from a disease.

The National Institute of Health (NIH) has published guidelines for the therapeutic management of adults with mild to moderate COVID or those at risk for progressing to severe COVID. See https://www.covid19treatmentguidelines.nih.gov/management/clinical-management-of-adults/nonhospitalized-adults-therapeutic-management/, last accessed on Apr. 10, 2023. For adults who are at high risk of progression for severe disease, the NIH notes that several antiviral therapeutic options are available to reduce the rise of hospitalization or death. The NIH's recommendation is reproduced in Table 3, below.

TABLE 3

| Patient Disposition | Panel's Recommendations |
|---|---|
| All Patients | All patients should be offered symptom management |
| | (Alll). |
| | The Panel recommends against the use of |
| | dexamethasone^{a} or other systemic corticosteroids |
| | in the absence of another indication (Allb). |
| Patients Who Are at High Risk of Progressing to | Preferred therapies. Listed in order of preference: |
| Severe COVID-19^{b} | Ritonavir-boosted nirmatrelvir (Paxlovid) (Alla) |
| | Remdesivir (Blla) |
| | Alternative therapy. For use when then preferred |
| | therapies are not available, feasible to use, or clinically |
| | appropriate: |
| | Monupiravir (Clla) |

In addition, as apparent to the skilled artisan, as therapies are developed to address new subvariants of the COVID virus, such as new antibody therapies similar to Remdesivir can be desired. In addition, those at risk of severe COVID or death should be vaccinated according to current and future guidelines, and boosted at regular intervals, such as every 6 months. These therapies can be combined with corticosteroids as appropriate.

Corticosteriods or steroids, are a class of drugs that are anti-inflammatory medicines. Corticosterioids include cortisone ((8S,9S,10R,13S,14S,17R)-17-hydroxy-17-(2-bydroxyacetyl)-10,13-dimethyl-1,2,6,7,8,9,12,14,15,16-decahydrocyclopenta[a]phenanthrene-3,11-dione), hydrocortisone (11β,17α,21-Trihydroxypregn-4-ene-3,20-dione; 11β,17α,21-Trihydroxyprogesterone) and prednisone (17α,21-Dihydroxypregn-4-ene-3,11,20-trione).

Dexamethasone is a long acting glucocorticoid. It is sold under the brand names Bacycadron, Elixier, and Decadron. The chemical name is 9α-fluoro-llβ,17α,21-trihydroxy-16α-methylpregna-1,4-diene-3,2O-dione. Other names for the compound include 9α-fluoro-16α-methylprednisolone and 16α-methyl-9α-fluoroprednisolone.

Paxlovid is an oral medication for the treatment of COVID. It is also known as nirmatrelvir/ritonavir and commercially available from Pfizer.

Remdesivir sold under the tradename Veklury is a broad spectrum antiviral mediciation and commercially available from Gilead Sciences. The NIH describes the drug as a carboxylic ester resulting from the formal condensation of the carboxy group of N—[(S)-{[(2R,3S,4R,5R)-5-(4-aminopyrrolo[2,1-f][1,2,4]triazin-7-yl)-5-cyano-3,4-dihydroxytetrahydrofuran-2-yl]methoxy}(phenoxy)phosphoryl]-L-alanine with the hydroxy group of 2-ethylbutan-ethylbutan-1-ol. See https://pubchem.ncbi.nlm.nih.gov/compound/Remdesivir, last accessed on Apr. 10, 2023.

Molnupiravir is the oral prodrug of beta-D-N4-hydroxycytidine (NHC), a ribonucleoside that has shown antiviral activity against SARS-CoV-2 in vitro and in clinical trials. It is sold under the tradename Lagevrio. It is commercially available from Merck Sharp & Dohme Corp.

Vaccines for COVID are commercially available from Moderna (sold under the tradename SPIKEVAX), Pfizer-BioNTech, Novavaox and Johnson & Johnson.

As used herein, the term “sample” and “biological sample” are used interchangeably, referring to sample material derived from a subject. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples may include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears.

In some embodiments, the sample may be an upper respiratory specimen, such as a nasopharyngeal (NP) specimen, an oropharyngeal (OP) specimen, a nasal mid-turbinate swab, an anterior nares (nasal swab) specimen, or nasopharyngeal wash/aspirate or nasal wash/aspirate (NW) specimen.

In some embodiments, the samples include fluid from a subject, including, without limitation, blood or a blood product (e.g., serum, plasma, or the like), umbilical cord blood, amniotic fluid, cerebrospinal fluid, spinal fluid, lavage fluid (e.g., bronchoalveolar, gastric, peritoneal, ductal, ear, arthroscopic), washings of female reproductive tract, urine, feces, sputum, saliva, nasal mucous, prostate fluid, lavage, semen, lymphatic fluid, bile, tears, sweat, breast milk, breast fluid, the like or combinations thereof. In some embodiments, a liquid biological sample is a blood plasma or serum sample. The term “blood” as used herein refers to a blood sample or preparation from a subject. The term encompasses whole blood, blood product or any fraction of blood, such as serum, plasma, buffy coat, or the like as conventionally defined. In some embodiments, the term “blood” refers to peripheral blood. Blood plasma refers to the fraction of whole blood resulting from centrifugation of blood treated with anticoagulants. Blood serum refers to the watery portion of fluid remaining after a blood sample has coagulated. Fluid samples often are collected in accordance with standard protocols hospitals or clinics generally follow. For blood, an appropriate amount of peripheral blood (e.g., between 3-40 milliliters) often is collected and can be stored according to standard procedures prior to or after preparation.

A “patient” as used herein intends an animal patient, a mammal patient or yet further a human patient. For the purpose of illustration only, a mammal includes but is not limited to a simian, a murine, a bovine, an equine, a porcine or an ovine subject.

The term “suitable for a therapy” or “suitably treated with a therapy” shall mean that the patient is likely to exhibit one or more desirable clinical outcomes as compared to patients having the same disease and receiving the same therapy but possessing a different characteristic that is under consideration for the purpose of the comparison. In one aspect, the characteristic under consideration is a genetic polymorphism or a somatic mutation. In another aspect, the characteristic under consideration is expression level of a gene or a polypeptide. In one aspect, a more desirable clinical outcome is relatively higher likelihood of or relatively better outcome upon COVID infection.

As defined herein, the patient is considered suitable for the therapy. In another such aspect, a patient possessing a characteristic can exhibit one or more desirable clinical outcome but simultaneously exhibit one or more less desirable clinical outcome. The clinical outcomes can then be considered collectively, and a decision as to whether the patient is suitable for the therapy can be made accordingly, taking into account the patient's specific situation and the relevance of the clinical outcomes.

The term “identify” or “identifying” is to associate or affiliate a patient closely to a group or population of patients who likely experience the same or a similar clinical response to a therapy.

The term “selecting” a patient for a therapy refers to making an indication that the selected patient is suitable for the therapy. Such an indication can be made in writing by, for instance, a handwritten prescription or a computerized report making the corresponding prescription or recommendation.

When a genetic marker or polymorphism “is used as a basis” for identifying or selecting a patient for a treatment described herein, the genetic marker or polymorphism is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits; or (h) toxicity. As would be well understood by one in the art, measurement of the genetic marker or polymorphism in a clinical setting is a clear indication that this parameter was used as a basis for initiating, continuing, adjusting and/or ceasing administration of the treatments described herein.

The term “amplification” or “amplify” as used herein means one or more methods known in the art for copying a target nucleic acid, thereby increasing the number of copies of a selected nucleic acid sequence. Amplification can be exponential or linear. A target nucleic acid can be either DNA or RNA. The sequences amplified in this manner form an “amplicon.” While the exemplary methods described hereinafter relate to amplification using the polymerase chain reaction (“PCR”), numerous other methods are known in the art for amplification of nucleic acids (e.g., isothermal methods, rolling circle methods, etc.). The skilled artisan can understand that these other methods can be used either in place of, or together with, PCR methods.

The term “complement” as used herein means the complementary sequence to a nucleic acid according to standard Watson/Crick base pairing rules. A complement sequence can also be a sequence of RNA complementary to the DNA sequence or its complement sequence, and can also be a cDNA. The term “substantially complementary” as used herein means that two sequences hybridize under stringent hybridization conditions. The skilled artisan will understand that substantially complementary sequences need not hybridize along their entire length. In particular, substantially complementary sequences comprise a contiguous sequence of bases that do not hybridize to a target or marker sequence, positioned 3′ or 5′ to a contiguous sequence of bases that hybridize under stringent hybridization conditions to a target or marker sequence.

As used herein, the term “hybridize” or “specifically hybridize” refers to a process where two complementary nucleic acid strands anneal to each other under appropriately stringent conditions. Hybridizations are typically conducted with probe-length nucleic acid molecules. Nucleic acid hybridization techniques are well known in the art. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology. John Wiley & Sons, Secaucus, N.J.

“Primer” as used herein refers to an oligonucleotide that is capable of acting as a point of initiation of synthesis when placed under conditions in which primer extension is initiated (e.g., primer extension associated with an application such as PCR). The primer is complementary to a target nucleotide sequence and it hybridizes to a substantially complementary sequence in the target and leads to addition of nucleotides to the 3′-end of the primer in the presence of a DNA or RNA polymerase. The 3′-nucleotide of the primer should generally be complementary to the target sequence at a corresponding nucleotide position for optimal expression and amplification. An oligonucleotide “primer” can occur naturally, as in a purified restriction digest or can be produced synthetically. The term “primer” as used herein includes all forms of primers that can be synthesized including, peptide nucleic acid primers, locked nucleic acid primers, phosphorothioate modified primers, labeled primers, and the like.

Primers are typically between about 5 and about 100 nucleotides in length, such as between about 15 and about 60 nucleotides in length, such as between about 20 and about 50 nucleotides in length, such as between about 25 and about 40 nucleotides in length. In some embodiments, primers can be at least 8, at least 12, at least 16, at least 20, at least 25, at least 30, at least 35, at least 40, at least 45, at least 50, at least 55, at least 60 nucleotides in length. An optimal length for a particular primer application can be readily determined in the manner described in H. Erlich, PCR Technology. Principles and Application for DNA Amplification (1989).

“Probe” as used herein refers to nucleic acid that interacts with a target nucleic acid via hybridization. A probe can be fully complementary to a target nucleic acid sequence or partially complementary. The level of complementarity will depend on many factors based, in general, on the function of the probe. A probe or probes can be used, for example to detect the presence or absence of a mutation in a nucleic acid sequence by virtue of the sequence characteristics of the target. Probes can be labeled or unlabeled, or modified in any of a number of ways well known in the art. A probe can specifically hybridize to a target nucleic acid.

Probes can be DNA, RNA or a RNA/DNA hybrid. Probes can be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes can comprise modified nucleobases, modified sugar moieties, and modified internucleotide linkages. A probe can be fully complementary to a target nucleic acid sequence or partially complementary. A probe can be used to detect the presence or absence of a target nucleic acid. Probes are typically at least about 10, 15, 21, 25, 30, 35, 40, 50, 60, 75, 100 nucleotides or more in length.

“Detecting” as used herein refers to determining the presence of a nucleic acid of interest in a sample or the presence of a protein of interest in a sample. Detection does not require the method to provide 100% sensitivity and/or 100% specificity.

“Detectable label” as used herein refers to a molecule or a compound or a group of molecules or a group of compounds used to identify a nucleic acid or protein of interest. In some cases, the detectable label can be detected directly. In other cases, the detectable label can be a part of a binding pair, which can then be subsequently detected. Signals from the detectable label can be detected by various means and will depend on the nature of the detectable label. Detectable labels can be isotopes, fluorescent moieties, colored substances, and the like. Examples of means to detect detectable label include but are not limited to spectroscopic, photochemical, biochemical, immunochemical, electromagnetic, radiochemical, or chemical means, such as fluorescence, chemifluorescence, or chemiluminescence, or any other appropriate means.

“TaqMan® PCR detection system” as used herein refers to a method for real time PCR. In this method, a TaqMan® probe which hybridizes to the nucleic acid segment amplified is included in the PCR reaction mix. The TaqMan® probe comprises a donor and a quencher fluorophore on either end of the probe and in close enough proximity to each other so that the fluorescence of the donor is taken up by the quencher. However, when the probe hybridizes to the amplified segment, the 5′-exonuclease activity of the Taq polymerase cleaves the probe thereby allowing the donor fluorophore to emit fluorescence which can be detected.

As used herein, the term “sample” or “test sample” refers to any liquid or solid material containing nucleic acids. In suitable embodiments, a test sample is obtained from a biological source (i.e., a “biological sample”), such as cells in culture or a tissue sample from an animal, preferably, a human. In an exemplary embodiment, the sample is a biopsy sample.

“Target nucleic acid” as used herein refers to segments of a chromosome, a complete gene with or without intergenic sequence, segments or portions a gene with or without intergenic sequence, or sequence of nucleic acids to which probes or primers are designed. Target nucleic acids can include wild type sequences, nucleic acid sequences containing mutations, deletions or duplications, tandem repeat regions, a gene of interest, a region of a gene of interest or any upstream or downstream region thereof. Target nucleic acids can represent alternative sequences or alleles of a particular gene. Target nucleic acids can be derived from genomic DNA, cDNA, or RNA. As used herein, target nucleic acid can be native DNA or a PCR-amplified product.

As used herein the term “stringency” is used in reference to the conditions of temperature, ionic strength, and the presence of other compounds, under which nucleic acid hybridizations are conducted. With high stringency conditions, nucleic acid base pairing will occur only between nucleic acids that have sufficiently long segments with a high frequency of complementary base sequences. Exemplary hybridization conditions are as follows. High stringency generally refers to conditions that permit hybridization of only those nucleic acid sequences that form stable hybrids in 0.018 M NaCl at 65° C. High stringency conditions can be provided, for example, by hybridization in 50% formamide, 5×Denhardt's solution, 5×SSC (saline sodium citrate) 0.2% SDS (sodium dodecyl sulfate) at 42° C., followed by washing in 0.1×SSC, and 0.1% SDS at 65° C. Moderate stringency refers to conditions equivalent to hybridization in 50% formamide, 5×Denhardt's solution, 5×SSC, 0.2% SDS at 42° C., followed by washing in 0.2×SSC, 0.2% SDS, at 65° C. Low stringency refers to conditions equivalent to hybridization in 10% formamide, 5×Denhardt's solution, 6×SSC, 0.2% SDS, followed by washing in 1° SSC, 0.2% SDS, at 50° C.

As used herein the term “substantially identical” refers to a polypeptide or nucleic acid exhibiting at least 50%, 75%, 85%, 90%, 95%, or even 99% identity to a reference amino acid or nucleic acid sequence over the region of comparison. For polypeptides, the length of comparison sequences will generally be at least 20, 30, 40, or 50 amino acids or more, or the full length of the polypeptide. For nucleic acids, the length of comparison sequences will generally be at least 10, 15, 20, 25, 30, 40, 50, 75, or 100 nucleotides or more, or the full length of the nucleic acid.

### Descriptive Embodiments

The disclosure further provides diagnostic, prognostic and therapeutic methods, which are based, at least in part, on determination of the identify of a genotype of interest identified herein.

For example, information obtained using the diagnostic assays described herein is useful for determining if a subject is suitable for COVID treatment of a given type. Based on the prognostic information, a doctor can recommend a therapeutic protocol, useful for reducing the disease severity.

A patient's likely clinical outcome following a clinical procedure such as a therapy can be expressed in relative terms. For example, a patient having a particular genotype or expression level can experience relatively lower disease severity than a patient or patients not having the genotype or expression level or at higher risk for progressing to severe COVID.

It is to be understood that information obtained using the diagnostic assays described herein can be used alone or in combination with other information, such as, but not limited to, genotypes or expression levels of other genes, clinical chemical parameters, histopathological parameters, or age, gender and weight of the subject. When used alone, the information obtained using the diagnostic assays described herein is useful in determining or identifying the clinical outcome of a treatment, selecting a patient for a treatment, or treating a patient, etc. When used in combination with other information, on the other hand, the information obtained using the diagnostic assays described herein is useful in aiding in the determination or identification of clinical outcome of a treatment, aiding in the selection of a patient for a treatment, or aiding in the treatment of a patient and etc. In a particular aspect, the genotypes of one or more genes as disclosed herein are used in a panel of genes, each of which contributes to the final diagnosis, prognosis or treatment.

The methods are useful in the assistance of an animal, a mammal or yet further a human patient. For the purpose of illustration only, a mammal includes but is not limited to a human, a simian, a murine, a bovine, an equine, a porcine or an ovine subject.

### Diagnostic Methods

Applicant provides herein a method for identifying whether a patient is at risk for progressing to severe COVID comprising, or consisting of, or consisting of screening a biological sample isolated from the patient for 1 or more polymorphisms selected from the group shown in Table 1, and identifying that the patient is at risk for progressing to severe COVID if 1 or more polymorphisms are identified in the sample. In alternative aspects, wherein 2 or more, or 3 or more, or all 4 polymorphisms are identified in the sample. In another embodiment, the polymorphormism is identified by a method comprising the use of the probes and primers shown in Appendix A.

In one aspect the method further comprises administering an effective amount of a COVID vaccine to the patient, such as for example a booster vaccine.

Also provided is a method for identifying whether a patient is not at risk for progressing to severe COVID comprising, or consisting essentially of, or yet further consisting of screening a biological sample isolated from the patient for 1 or more polymorphisms selected from the group shown in Table 1, and identifying that the patient is not at risk for progressing to severe COVID if none of the polymorphisms are identified in the sample. In alternative aspects, wherein 2 or more, or 3 or more, or all 4 polymorphisms are identified in the sample. In another embodiment, the polymorphormism is identified by a method comprising the use of the probes and primers shown in Appendix A.

In one aspect, the patient is a human patient.

Alternatively, the screening comprises contacting the biological sample or nucleic acid isolated from the biological sample with a labeled nucleic acid probe that specifically binds to a polymorphic region of interest having the polymorphic region, optionally a probe and primer pair as shown in Appendix A for each relevant SNP. The sample is at least one of blood, plasma, an original sample recently isolated from the patient, a peripheral blood lymphocyte, a fixed tissue, a frozen tissue, a biopsy tissue, a resection tissue, a microdissected tissue, or combinations thereof.

The screening can be performed by a method comprising PCR, PCR-RFLP, whole genome sequencing, sequencing, or microarray.

Also provided is probe and primer pair as shown in Appendix A and optionally preservative and/or a stabilizer for the probes and primer pairs. Non-limiting examples of stabilizers and preservatives include trehalose (TRH), gelatine (GEL) and polyethylene glycol (PEG) See Zagon et al. (2012) Food Control Vol. 25, Issue 2, pages 709-716.

Also provided is a kit for performing the methods as described herein, comprising reagents to identify or determine the genotype of the sample and instructions for use.

Yet further provided is a method for detecting a SNP identified in Table 1 in a biological sample isolated from a patient, comprising screening the biological sample for one more SNPs identified in Table 1.

Any suitable method for identifying the genotype in the patient sample can be used and the disclosures described herein are not to be limited to these methods. For the purpose of illustration only, the genotype is determined by a method comprising, or alternatively consisting essentially of, or yet further consisting of, sequencing, hybridization, nucleic acid amplification, including polymerase chain reaction (PCR), real-time PCR, reverse transcriptase PCR (RT-PCR), nested PCR, ligase chain reaction, whole genome sequencing, or PCR-RFLP, or microarray. These methods as well as equivalents or alternatives thereto are described herein.

Prior art methods can be used to detect and identify the SNPs of Table 1 or such can be designed using methods known in the art.

The methods are useful in the assistance of an animal, a mammal or yet further a human patient. For the purpose of illustration only, a mammal includes but is not limited to a human, a simian, a murine, a bovine, an equine, a porcine or an ovine subject.

Information obtained using the diagnostic assays described herein is useful for determining if a subject will likely, more likely, or less likely to experience mild, moderate or severe COVID. Based on the prognostic information, a doctor can recommend a therapeutic protocol, useful for treating reducing disease severity in the patient. A patient having a sample with 2 or more, or 3 or more or 4 or more is more likely to experience a progression to severe disease.

In addition, knowledge of the identity of a particular allele in an individual (the gene profile) allows customization of therapy for a particular disease to the individual's genetic profile, the goal of “pharmacogenomics”. For example, an individual's genetic profile can enable a doctor: 1) to more effectively prescribe a drug that will address the molecular basis of the disease or condition; 2) to better determine the appropriate dosage of a particular drug and 3) to identify novel targets for drug development. The identity of the genotype or expression patterns of individual patients can then be compared to the genotype or expression profile of the disease to determine the appropriate drug and dose to administer to the patient.

The ability to target populations expected to show the highest clinical benefit, based on the normal or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling.

### Biological Sample Collection and Preparation

The methods and compositions disclosed herein can be used to detect nucleic acids associated with the genetic polymorphisms identified herein and in particular those noted in Table A using a biological sample obtained from a patient. Biological samples can be obtained by standard procedures and can be used immediately or stored, under conditions appropriate for the type of biological sample, for later use. Any liquid or solid biological material obtained from the patient believed to contain nucleic acids comprising the region the polymorphic region can be an suitable sample.

Methods of obtaining test samples are known to those of skill in the art and include, but are not limited to, aspirations, tissue sections, swabs, drawing of blood or other fluids, surgical or needle biopsies.

In some aspects, the biological sample is a tissue or a cell sample. Suitable patient samples in the methods include, but are not limited to, blood, plasma, serum, a biopsy tissue, fine needle biopsy sample, amniotic fluid, plasma, peripheral blood lymphocytes (PBLs), pleural fluid, saliva, semen, serum, tissue or tissue homogenates, frozen or paraffin sections of tissue or combinations thereof. In some aspects, the biological sample is an original sample recently isolated from the patient, a fixed tissue, a frozen tissue, a resection tissue, or a microdissected tissue. In some aspects, the biological samples are processed, such as by sectioning of tissues, fractionation, purification, nucleic acid isolation, or cellular organelle separation.

In some embodiments, nucleic acid (DNA or RNA) is isolated from the sample according to any methods known to those of skill in the art. In some aspects, genomic DNA is isolated from the biological sample. In some aspects, RNA is isolated from the biological sample. In some aspects, cDNA is generated from mRNA in the sample. In some embodiments, the nucleic acid is not isolated from the biological sample (e.g., the polymorphism is detected directly from the biological sample).

### Detection of Polymorphisms

In some aspects, detection of polymorphisms can be accomplished by molecular cloning of the specified allele and subsequent sequencing of that allele using techniques known in the art, in some aspects, after isolation of a suitable nucleic acid sample. In some aspects, the gene sequences can be amplified directly from a genomic DNA preparation from the biological sample using PCR, and the sequence composition is determined by sequencing the amplified product (i.e., amplicon). Alternatively, the PCR product can be analyzed following digestion with a restriction enzyme, a method known as PCR-RFLP. Yet further, real time PCR is used to determine the phenotype of the gene of interest, using methods known in the art. See, https://www.bio-rad.com/en-us/applications-technologies/what-real-time-pcr-qpcr?ID=LUSO4W8UU, last accessed on Apr. 9, 2023.

In some embodiments, the polymorphism is detected using allele specific hybridization using probes overlapping the polymorphic site. In some aspects, the nucleic acid probes are between 5 and 40 nucleotides in length. In some aspects, the nucleic acid probes are about 5, about 10, about 15, about 20, about 25, about 30, about 35, or about 40 or more nucleotides flanking the polymorphic site.

In another embodiment of the disclosure, several nucleic acid probes capable of hybridizing specifically to the nucleic acid containing the allelic variant are attached to a solid phase support, e.g., a “chip” or “microarray. Such gene chips or microarrays can be used to detect genetic variations by a number of techniques known to one of skill in the art. In one technique, oligonucleotides are arrayed on a gene chip for determining the DNA sequence by the sequencing by hybridization approach. The probes of the disclosure also can be used for fluorescent detection of a genetic sequence. A probe also can be affixed to an electrode surface for the electrochemical detection of nucleic acid sequences.

In one aspect, “gene chips” or “microarrays” containing probes or primers for the gene of interest are provided alone or in combination with other probes and/or primers. A suitable sample is obtained from the patient extraction of genomic DNA, RNA, or any combination thereof and amplified if necessary. The DNA or RNA sample is contacted to the gene chip or microarray panel under conditions suitable for hybridization of the gene(s) of interest to the probe(s) or primer(s) contained on the gene chip or microarray. The probes or primers can be detectably labeled thereby identifying the polymorphism in the gene(s) of interest. Alternatively, a chemical or biological reaction can be used to identify the probes or primers which hybridized with the DNA or RNA of the gene(s) of interest. The genetic profile of the patient is then determined with the aid of the aforementioned apparatus and methods.

In some aspects, whole genome sequencing, in particular with the “next generation sequencing” techniques, which employ massively parallel sequencing of DNA templates, can be used to obtain genotypes of relevant polymorphisms. Exemplary NGS sequencing platforms for the generation of nucleic acid sequence data include, but are not limited to, Illumina's sequencing by synthesis technology (e.g., Illumina MiSeq or HiSeq System), Life Technologies' Ion Torrent semiconductor sequencing technology (e.g., Ion Torrent PGM or Proton system), the Roche (454 Life Sciences) GS series and Qiagen (Intelligent BioSystems) Gene Reader sequencing platforms.

In some aspects, nucleic acid comprising the polymorphism is amplified to produce an amplicon containing the polymorphism. Nucleic acids can be amplified by various methods known to the skilled artisan. Nucleic acid amplification can be linear or exponential. Amplification is generally carried out using polymerase chain reaction (PCR) technologies. Alternative or modified PCR amplification methods can also be used and include, for example, isothermal amplification methods, rolling circle methods, Hot-start PCR, real-time PCR, Allele-specific PCR, Assembly PCR or Polymerase Cycling Assembly (PCA), Asymmetric PCR, Colony PCR, Emulsion PCR, Fast PCR, Real-Time PCR, nucleic acid ligation, Gap Ligation Chain Reaction (Gap LCR), Ligation-mediated PCR, Multiplex Ligation-dependent Probe Amplification, (MLPA), Gap Extension Ligation PCR (GEXL-PCR), quantitative PCR (Q-PCR), Quantitative real-time PCR (QRT-PCR), multiplex PCR, Helicase-dependent amplification, Intersequence-specific (ISSR) PCR, Inverse PCR, Linear-After-The-Exponential-PCR (LATE-PCR), Methylation-specific PCR (MSP), Nested PCR, Overlap-extension PCR, PAN-AC assay, Reverse Transcription PCR (RT-PCR), Rapid Amplification of cDNA Ends (RACE PCR), Single molecule amplification PCR (SMA PCR), Thermal asymmetric interlaced PCR (TAIL-PCR), Touchdown PCR, long PCR, nucleic acid sequencing (including DNA sequencing and RNA sequencing), transcription, reverse transcription, duplication, DNA or RNA ligation, and other nucleic acid extension reactions known in the art. The skilled artisan will understand that other methods can be used either in place of, or together with, PCR methods, including enzymatic replication reactions developed in the future. See, e.g., Saiki, “Amplification of Genomic DNA” in PCR Protocols, Innis et al., eds., Academic Press, San Diego, Calif., 13-20 (1990); Wharam, et al., 29 (11) Nucleic Acids Res, E54-E54 (2001); Hafner, et al., 30 (4) Biotechniques, 852-6, 858, 860 passim (2001).

In some aspects, nucleic acid comprising the polymorphism of interest is amplified to produce an amplicon. In some aspects, a nucleic acid containing the region of interest is amplified using a forward primer and a reverse primer the flank the region of interest. In some aspects, the amplicon containing the region of interest (e.g. an amplicon having the polymorphic sequence) is detected by hybridizing a nucleic acid probe containing the polymorphism or a complement thereof to the corresponding complementary strand of the amplicon and detecting the hybrid formed between the nucleic acid probe and the complementary strand of the amplicon. In some aspects, amplicon containing the region of interest is sequenced (e.g., dideoxy chain termination methods (Sanger method and variants thereof), Maxam & Gilbert sequencing, pyrosequencing, exonuclease digestion and next-generation sequencing methods).

In some embodiments, the amplification includes a labeled primer or probe, thereby allowing detection of the amplification products corresponding to that primer or probe. In particular embodiments, the amplification can include a multiplicity of labeled primers or probes; such primers can be distinguishably labeled, allowing the simultaneous detection of multiple amplification products.

In some embodiments, the amplification products are detected by any of a number of methods such as gel electrophoresis, column chromatography, hybridization with a nucleic acid probe, or sequencing the amplicon.

Detectable labels can be used to identify the primer or probe hybridized to a genomic nucleic acid or amplicon. Detectable labels include but are not limited to fluorophores, isotopes (e.g., ³²P, ³³P, ³⁵S, ³H, ¹⁴C, ¹²⁵I, ¹³¹I) electron-dense reagents (e.g., gold, silver), nanoparticles, enzymes commonly used in an ELISA (e.g., horseradish peroxidase, beta-galactosidase, luciferase, alkaline phosphatase), chemiluminiscent compounds, colorimetric labels (e.g., colloidal gold), magnetic labels (e.g., Dynabeads®), biotin, digoxigenin, haptens, proteins for which antisera or monoclonal antibodies are available, ligands, hormones, oligonucleotides capable of forming a complex with the corresponding oligonucleotide complement.

In one embodiment, a primer or probe is labeled with a fluorophore that emits a detectable signal. The term “fluorophore” as used herein refers to a molecule that absorbs light at a particular wavelength (excitation frequency) and subsequently emits light of a longer wavelength (emission frequency). While a suitable reporter dye is a fluorescent dye, any reporter dye that can be attached to a detection reagent such as an oligonucleotide probe or primer is suitable for use in the methods described. Suitable fluorescent moieties include, but are not limited to, the following fluorophores working individually or in combination: 4-acetamido-4′-isothiocyanatostilbene-2,2′disulfonic acid; acridine and derivatives, e.g., acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescein (TET); fluorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron® Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAMRA); tetramethyl rhodamine; and tetramethyl rhodamine isothiocyanate (TRITC).

In some aspects, the primer or probe is further labeled with a quencher dye such as Tamra, Dabcyl, or Black Hole Quencher® (BHQ), especially when the reagent is used as a self-quenching probe such as a TaqMan® (U.S. Pat. Nos. 5,210,015 and 5,538,848) or Molecular Beacon probe (U.S. Pat. Nos. 5,118,801 and 5,312,728), or other stemless or linear beacon probe (Livak et al., 1995, PCR Method Appl., 4:357-362; Tyagi et al, 1996, Nature Biotechnology, 14:303-308; Nazarenko et al., 1997, Nucl. Acids Res., 25:2516-2521; U.S. Pat. Nos. 5,866,336 and 6,117,635).

In some aspects, methods for real time PCR use fluorescent primers/probes, such as the TaqMan® primers/probes (Heid, et al., Genome Res 6:986-994, 1996), molecular beacons, and Scorpion™ primers/probes. Real-time PCR quantifies the initial amount of the template with more specificity, sensitivity and reproducibility, than other forms of quantitative PCR, which detect the amount of final amplified product. Real-time PCR does not detect the size of the amplicon. The probes employed in Scorpion®™ and TaqMan® technologies are based on the principle of fluorescence quenching and involve a donor fluorophore and a quenching moiety. The term “donor fluorophore” as used herein means a fluorophore that, when in close proximity to a quencher moiety, donates or transfers emission energy to the quencher. As a result of donating energy to the quencher moiety, the donor fluorophore will itself emit less light at a particular emission frequency that it would have in the absence of a closely positioned quencher moiety. The term “quencher moiety” as used herein means a molecule that, in close proximity to a donor fluorophore, takes up emission energy generated by the donor and either dissipates the energy as heat or emits light of a longer wavelength than the emission wavelength of the donor. In the latter case, the quencher is considered to be an acceptor fluorophore. The quenching moiety can act via proximal (i.e., collisional) quenching or by Forster or fluorescence resonance energy transfer (“FRET”). Quenching by FRET is generally used in TaqMan® primers/probes while proximal quenching is used in molecular beacon and Scorpion™ type primers/probes.

The detectable label can be incorporated into, associated with or conjugated to a nucleic acid primer or probe. Labels can be attached by spacer arms of various lengths to reduce potential steric hindrance or impact on other useful or desired properties. See, e.g., Mansfield, Mol. Cell. Probes (1995), 9:145-156.

Detectable labels can be incorporated into nucleic acid probes by covalent or non-covalent means, e.g., by transcription, such as by random-primer labeling using Klenow polymerase, or nick translation, or, amplification, or equivalent as is known in the art. For example, a nucleotide base is conjugated to a detectable moiety, such as a fluorescent dye, e.g., Cy3™ or Cy5™ and then incorporated into nucleic acid probes during nucleic acid synthesis or amplification. Nucleic acid probes can thereby be labeled when synthesized using Cy3™- or Cy5™-dCTP conjugates mixed with unlabeled dCTP.

Nucleic acid probes can be labeled by using PCR or nick translation in the presence of labeled precursor nucleotides, for example, modified nucleotides synthesized by coupling allylamine-dUTP to the succinimidyl-ester derivatives of the fluorescent dyes or haptens (such as biotin or digoxigenin) can be used; this method allows custom preparation of most common fluorescent nucleotides, see, e.g., Henegariu et al., Nat. Biotechnol. (2000), 18:345-348,

Nucleic acid probes can be labeled by non-covalent means known in the art. For example, Kreatech Biotechnology's Universal Linkage System® (ULS®) provides a non-enzymatic labeling technology, wherein a platinum group forms a co-ordinative bond with DNA, RNA or nucleotides by binding to the N7 position of guanosine. This technology can also be used to label proteins by binding to nitrogen and sulfur containing side chains of amino acids. See, e.g., U.S. Pat. Nos. 5,580,990; 5,714,327; and 5,985,566; and European Patent No. 0539466.

Labeling with a detectable label also can include a nucleic acid attached to another biological molecule, such as a nucleic acid, e.g., an oligonucleotide, or a nucleic acid in the form of a stem-loop structure as a “molecular beacon” or an “aptamer beacon”. Molecular beacons as detectable moieties are described; for example, Sokol (Proc. Natl. Acad. Sci. USA (1998), 95:11538-11543) synthesized “molecular beacon” reporter oligodeoxynucleotides with matched fluorescent donor and acceptor chromophores on their 5′ and 3′ ends. In the absence of a complementary nucleic acid strand, the molecular beacon remains in a stem-loop conformation where fluorescence resonance energy transfer prevents signal emission. On hybridization with a complementary sequence, the stem-loop structure opens increasing the physical distance between the donor and acceptor moieties thereby reducing fluorescence resonance energy transfer and allowing a detectable signal to be emitted when the beacon is excited by light of the appropriate wavelength. See also, e.g., Antony (Biochemistry (2001), 40:9387-9395), describing a molecular beacon consist of a G-rich 18-mer triplex forming oligodeoxyribonucleotide. See also U.S. Pat. Nos. 6,277,581 and 6,235,504.

Aptamer beacons are similar to molecular beacons; see, e.g., Hamaguchi, Anal. Biochem. (2001), 294:126-131; Poddar, Mol. Cell. Probes (2001), 15:161-167; Kaboev, Nucleic Acids Res. (2000), 28: E94. Aptamer beacons can adopt two or more conformations, one of which allows ligand binding. A fluorescence-quenching pair is used to report changes in conformation induced by ligand binding. See also, e.g., Yamamoto et al., Genes Cells (2000), 5:389-396; Smimov et al., Biochemistry (2000), 39:1462-1468.

The nucleic acid primer or probe can be indirectly detectably labeled via a peptide. A peptide can be made detectable by incorporating predetermined polypeptide epitopes recognized by a secondary reporter (e.g., leucine zipper pair sequences, binding sites for secondary antibodies, transcriptional activator polypeptide, metal binding domains, epitope tags). A label can also be attached via a second peptide that interacts with the first peptide (e.g., S— association).

As readily recognized by one of skill in the art, detection of the complex containing the nucleic acid from a sample hybridized to a labeled probe can be achieved through use of a labeled antibody against the label of the probe. In one example, the probe is labeled with digoxigenin and is detected with a fluorescent labeled anti-digoxigenin antibody. In another example, the probe is labeled with FITC, and detected with fluorescent labeled anti-FITC antibody. These antibodies are readily available commercially. In another example, the probe is labeled with FITC, and detected with anti-FITC antibody primary antibody and a labeled anti-anti FITC secondary antibody.

Nucleic acids can be amplified prior to detection or can be detected directly during an amplification step (i.e., “real-time” methods, such as in TaqMan® and Scorpion™ methods). In some embodiments, the target sequence is amplified using a labeled primer such that the resulting amplicon is detectably labeled. In some embodiments, the primer is fluorescently labeled. In some embodiments, the target sequence is amplified and the resulting amplicon is detected by electrophoresis.

With regard to the exemplary primers and probes, those skilled in the art will readily recognize that nucleic acid molecules can be double-stranded molecules and that reference to a particular site on one strand refers, as well, to the corresponding site on a complementary strand. In defining a variant position, allele, or nucleotide sequence, reference to an adenine, a thymine (uridine), a cytosine, or a guanine at a particular site on one strand of a nucleic acid molecule also defines the thymine (uridine), adenine, guanine, or cytosine (respectively) at the corresponding site on a complementary strand of the nucleic acid molecule. Thus, reference can be made to either strand in order to refer to a particular variant position, allele, or nucleotide sequence. Probes and primers, can be designed to hybridize to either strand and detection methods disclosed herein can generally target either strand.

In some embodiments, the primers and probes comprise additional nucleotides corresponding to sequences of universal primers (e.g., T7, M13, SP6, T3) which add the additional sequence to the amplicon during amplification to permit further amplification and/or prime the amplicon for sequencing.

### Methods of Treatment

The disclosure further provides methods of treating a patient selected by any method of the above embodiments, or identified as likely to experience a more favorable clinical outcome by any of the above methods, following the therapy. In some embodiments, the methods entail administering to the patients such a therapy. The therapy can be any one of the group of: a corticosteroid, Paxlovid, Remdesivir, or Molnupiravir, or other equivalent therapy recommended for patients who are at high risk for progressing to severe COVID or death.

In some aspects, the patient is selected by a method comprising screening a tissue or cell sample isolated from the patient for a polymorphism identified in Table A or as disclosed herein. Exemplary methods for screening are described in the diagnostic methods provided above and throughout the present disclosure. Any such diagnostic methods disclosed for the detection of a genetic polymorphism can be combined with the treatment methods provided herein.

The methods are useful in the assistance of an animal, a mammal or yet further a human patient. For the purpose of illustration only, a mammal includes but is not limited to a human, a simian, a murine, a bovine, an equine, a porcine or an ovine subject. Accordingly, a formulation comprising the necessary therapy or equivalent thereof is further provided herein. The formulation can further comprise one or more preservatives or stabilizers.

The agents or drugs can be administered as a composition. A “composition” typically intends a combination of the active agent and another carrier, e.g., compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates.

The agents identified herein as effective for their intended purpose can be administered to subjects or individuals identified by the methods herein as suitable for the therapy. Therapeutic amounts can be empirically determined and will vary with the pathology being treated, the subject being treated and the efficacy and toxicity of the agent.

Methods of administering pharmaceutical compositions are well known to those of ordinary skill in the art and include, but are not limited to, oral, microinjection, intravenous or parenteral administration. The compositions are intended for topical, oral, or local administration as well as intravenously, subcutaneously, or intramuscularly. Administration can be effected continuously or intermittently throughout the course of the treatment. Methods of determining the most effective means and dosage of administration are well known to those of skill in the art and will vary with the infection being treated and the patient and the subject being treated. Single or multiple administrations can be carried out with the dose level and pattern being selected by the treating physician.

### Kits

Kits or panel for use in detecting the polymorphism of interest in patient biological samples are provided. In some embodiments, a kit comprises at least one reagent necessary to perform the assay. For example, the kit can comprise an enzyme, a buffer or any other necessary reagent (e.g. PCR reagents and buffers). For example, in some aspects, a kit contains, in an amount sufficient for at least one assay, any of the hybridization assay probes, amplification primers, and/or antibodies suitable for detection in a packaging material.

The various components of the kit can be provided in a variety of forms. For example, in some aspects, the required enzymes, the nucleotide triphosphates, the probes, primers, and/or antibodies are be provided as a lyophilized reagent. These lyophilized reagents can be pre-mixed before lyophilization so that when reconstituted they form a complete mixture with the proper ratio of each of the components ready for use in the assay. In addition, the kits can contain a reconstitution reagent for reconstituting the lyophilized reagents of the kit. In exemplary kits for amplifying target nucleic acid, the enzymes, nucleotide triphosphates and required cofactors for the enzymes are provided as a single lyophilized reagent that, when reconstituted, forms a proper reagent for use in the present amplification methods.

Typically, the kits will also include instructions recorded in a tangible form (e.g., contained on paper or an electronic medium) for using the packaged probes, primers, and/or antibodies in a detection assay for determining the presence or amount of the polymorphism of interest in a test sample.

In some aspects, the kits further comprise a solid support for anchoring the nucleic acid of interest on the solid support. The target nucleic acid can be anchored to the solid support directly or indirectly through a capture probe anchored to the solid support and capable of hybridizing to the nucleic acid of interest. Examples of such solid support include but are not limited to beads, microparticles (for example, gold and other nano particles), microarray, microwells, multiwell plates. The solid surfaces can comprise a first member of a binding pair and the capture probe or the target nucleic acid can comprise a second member of the binding pair. Binding of the binding pair members will anchor the capture probe or the target nucleic acid to the solid surface. Examples of such binding pairs include but are not limited to biotin/streptavidin, hormone/receptor, ligand/receptor, and antigen/antibody.

The kit can comprise at least one probe or primer which is capable of specifically hybridizing to the gene of interest and instructions for use, e.g., those provided in Appendix A. For example, in some aspects, the kits comprise at least one of the above described nucleic acids. Exemplary kits for amplifying at least a portion of the gene of interest comprise two primers. For example, in some embodiments, the kit comprises a forward primer and a reverse primer that flank the polymorphism.

In some embodiments, the kit further comprises a nucleic acid probe for the detection of the amplicon. In some embodiments, the nucleic acid probe has about 5, about 10, about 15, about 20, or about 25, or about 30, about 35, about 40 or more contiguous nucleotides. In some aspects, the nucleic acid primers and/or probes are lyophilized.

In some embodiments, at least one of the primers for amplification is capable of hybridizing to the allelic variant sequence. For example, in some embodiments, at least one of the primers for amplification has about 5, about 10, about 15, about 20, or about 25, or about 30, about 35, about 40 or more contiguous nucleotides of any of genetic region that flank the polymorphism of interest. Such kits are suitable for detection of genotype by, for example, fluorescence detection, by electrochemical detection, or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the surface is silica or glass. In another embodiment, the surface is a metal electrode.

The kits can include all or some of the positive controls, negative controls, reagents, primers, sequencing markers, probes and antibodies described herein for determining the subject's genotype in the polymorphic region of the gene of interest or target region.

As amenable, these suggested kit components can be packaged in a manner customary for use by those of skill in the art. For example, these suggested kit components can be provided in solution or as a liquid dispersion or the like.

Typical packaging materials would include solid matrices such as glass, plastic, paper, foil, micro-particles and the like, capable of holding within fixed limits hybridization assay probes, and/or amplification primers. Thus, for example, the packaging materials can include glass vials used to contain sub-milligram (e.g., picogram or nanogram) quantities of a contemplated probe, primer, or antibodies or they can be microtiter plate wells to which probes, primers, or antibodies have been operatively affixed, i.e., linked so as to be capable of participating in an amplification and/or detection methods.

The instructions will typically indicate the reagents and/or concentrations of reagents and at least one assay method parameter which might be, for example, the relative amounts of reagents to use per amount of sample. In addition, such specifics as maintenance, time periods, temperature, and buffer conditions can also be included.

The diagnostic systems contemplate kits having any of the hybridization assay probes, amplification primers, or antibodies described herein, whether provided individually or in one of the combinations described above, for use in determining the presence or amount of a polymorphism of interest, e.g. noted in Table 4 or as identified herein.

The disclosure now being generally described, it will be more readily understood by reference to the following example which is included merely for purposes of illustration of certain aspects and embodiments of the present disclosure, and are not intended to limit the disclosure.

### Distinguish Between WT and MT Sequences

Applicant can validate the custom TaqMan SNP assays to distinguish between the WT and MT sequence and that they do not recognize the other sequences. Each assay can be run using 5 ng of control plasmid template DNA. Control plasmids can be prepared as WT alone, MT alone and a 1:1 mix of WT and MT. Each TaqMan assay can be run on each control plasmid and WT: MT mix. Each assay can be run a minimum of 5 times with each assay control plasmid and repeated by a second technician independently at separate times. A non-template control (NTC) reaction can be run for each assay to determine the background Ct value. If any assay gives a Ct value (≥10 or ≤NTC Ct value) for a control plasmid that is not the control plasmid for that assay, the assay can be re-designed to create a specific assay.

### Sensitivity

To determine the minimum amount of template DNA that can be reliably detected by each assay (limit of detection or sensitivity), each assay can be run using control plasmid DNA for WT and MT but not the WT: MT mix. DNA concentrations of 5, 2.5, 1, 0.5, 0.1 and 0.05 ng per reaction can be used. Each assay can be run a minimum of 5 times and repeated by a second technician independently at separate times. The limit of detection shall be the lowest amount of plasmid DNA per reaction detected by all 6 assays with Ct values>NTC Ct value.

### Reproducibility and Accuracy

Once the limit of detection has been determined, each assay can be run with the control plasmids for WT alone, MT alone and WT: MT mix at the limit of detection plus 0.5 ng, 5 times consecutively with 1-2 days between each qPCR run. Two technicians can carry out these 5 qPCR runs at separate times. The Assays can be considered reproducible if Ct values obtained for the WT alone, MT alone and WT: MT mix for each assay run display a variation of ≤5% for each technician and between technicians.

Genome Explorations can source gDNA (or total RNA from which cDNA can be prepared) from up to 20 patients who have been tested for the SARS-CoV-2 virus. These 20 patients can consist of patients who tested both positive and negative. Each sample can be tested by two technicians a minimum of 1-3 times (depending on sample availability) and the test considered accurate and reproducible if the variation in Ct values between technicians is ≤5%.

Finally, the Ct values obtained for each qPCR run for WT alone, Mt alone and WT: MT, where applicable, for each separate technician can be compared and the assays considered accurate if the variation in Ct values between technicians is ≤5%.

### Platform Validation

Patient samples can also be run on a BioRad CFX384 real-time PCR system in addition to the AB7900HT. If the results obtained have Ct values that vary from those obtained using the AB7900HT real-time PCR system by ≤5%, the AB7900HT platform can be considered to validated as the platform for performing the assays. If the BioRad CFX384 produces results within the same range, this can be considered as validated as a second real-time platform for running this assay.

### Technical Validation

In order to determine if the assays are detecting the correct SNP in patient samples, the sourced samples can be subjected to Sanger or NGS sequencing to determine what the genotype of the sample was. If these two approaches provide the same answer then the assay can be considered to have been technically validated.

The COVID-19 HGI identified 4 genome-wide SNPs that were directly related to hospitalization and death due to COVID-19.

### EQUIVALENTS

The disclosure illustratively described herein can suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by preferred embodiments and optional features, modification, improvement and variation of the disclosure embodied therein herein disclosed can be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art can recognize that the disclosure is also thereby described in terms of any individual member or subgroup of members of the Markush group.

All publications, patent applications, patents, and other references mentioned herein are expressly incorporated by reference in their entirety, to the same extent as if each were incorporated by reference individually. Several references are identified by an Arabic number, and the full bibliographic citation or these references are provided below. In case of conflict, the present specification, including definitions, will control.

### REFERENCES

[1] The covid-19 host genetics initiative, a global initiative to elucidate the role of host genetic factors in susceptibility and severity of the SARS-COV-2 virus pandemic. (2020). European Journal of Human Genetics, 28 (6), 715-718. https://doi.org/10.1038/s41431-020-0636-6
[2] Ganna, A. (2021). Mapping the human genetic architecture of COVID-19: An update. MedRxiv. https://doi.org/10.1101/2021.11.08.21265944
[3] COVID-19 Host Genetics Initiative. Mapping the human genetic architecture of COVID-19. Nature 600, 472-477 (2021). https://doi.org/10.1038/s41586-021-03767-x
[4] Genomewide Association study of severe COVID-19 with respiratory failure. (2020). New England Journal of Medicine, 383 (16), 1522-1534. https://doi.org/10.1056/nejmoa2020283
[5] Horowitz, J. E., Kosmicki, J. A., Damask, A. et al. Genome-wide analysis provides genetic evidence that ACE2 influences COVID-19 risk and yields risk scores associated with severe disease. Nat Genet 54, 382-392 (2022). https://doi.org/10.1038/s41588-021-01006-7
[6] Kousathanas, A., Pairo-Castineira, E., Rawlik, K. et al. Whole genome sequencing reveals host factors underlying critical Covid-19. Nature (2022). https://doi.org/10.1038/s41586-022-04576-6
[7] Pairo-Castineira, E., Clohisey, S., Klaric, L. et al. Genetic mechanisms of critical illness in COVID-19. Nature 591, 92-98 (2021). https://doi.org/10.1038/s41586-020-03065-y
[8] Roberts, G. H. L., Park, D. S., Coignet, M. V., McCurdy, S. R., Knight, S. C., Partha, R., Rhead, B., Zhang, M., Berkowitz, N., Haug Baltzell, A. K., Guturu, H., Girshick, A. R., Rand, K. A., Hong, E. L., & Ball, C. A. (2020). Ancestrydna Covid-19 host genetic study identifies three novel loci. Medrxiv. https://doi.org/10.1101/2020.10.06.20205864
[9] Roberts, G. H. L., Partha, R., Rhead, B., Knight, S. C., Park, D. S., Coignet, M. V., Zhang, M., Berkowitz, N., Turrisini, D. A., Gaddis, M., McCurdy, S. R., Pavlovic, M., Ruiz, L., Baltzell, A. K., Guturu, H., Girshick, A. R., Rand, K. A., Hong, E. L., & Ball, C. A. (2021). Novel covid-19 phenotype definitions reveal phenotypically distinct patterns of Genetic Association and protective effects. MedRxiv. https://doi.org/10.1101/2021.01.24.21250324
[10] Shelton, J. F., Shastri, A. J., Ye, C., Weldon, C. H., Filshtein-Somnez, T., Coker, D., Symons, A., Esparza-Gordillo, J., Aslibekyan, S., & Auton, A. (2020). Trans-ethnic analysis reveals genetic and non-genetic associations with covid-19) susceptibility and severity. MedRxiv https://doi.org/10.1101/2020.09.04.20188318

1. A method for identifying whether a patient is at risk for progressing to severe COVID comprising screening a biological sample isolated from the patient for 1 or more polymorphisms selected from the group shown in Table 1, and identifying that the patient is at risk for progressing to severe COVID if 1 or more polymorphisms are identified in the sample.

2. The method of claim 1, wherein 2 or more polymorphisms are identified in the sample.

3. The method of claim 1, wherein 3 or more polymorphisms are identified in the sample.

4. The method of claim 1, wherein all 4 polymorphisms are identified in the sample.

5. The method of claim 1, wherein the polymorphormism is identified by a method comprising the use of the probes and primers shown in Appendix A.

6. The method of claim 1, further comprising administering an effective amount of a COVID vaccine to the patient.

7. The method of claim 6, wherein the effective amount of the vaccine is a booster vaccine.

8. A method for identifying whether a patient is not at risk for progressing to severe COVID comprising screening a biological sample isolated from the patient for or more polymorphisms selected from the group shown in Table 1, and identifying that the patient is not at risk for progressing to severe COVID if none of the polymorphisms are identified in the sample.

9. The method of claim 8, wherein 1 or more polymorphisms are identified in the sample and the patient is identified as at risk for progressing to severe COVID.

10. A method for treating a patient likely to progress to severe COVID infection comprising administering an effective amount of a therapy selected from a COVID vaccine, a COVID booster, a corticosteroid, Paxlovid, Remdesivir, or Molnupiravir, or other equivalent therapy recommended for patients who are at high risk for progressing to severe COVID, wherein the patient has been identified for the treatment by screening a biological sample isolated from the patient for 1 or more polymorphisms selected from the group of Table 1, and identifying that the patient is at risk for progressing to severe COVID if 1 or more polymorphisms are identified in the sample.

11. The method of claim 10, wherein 3 or more polymorphisms are identified in the sample.

12. The method of claim 10, wherein 3 or more polymorphisms are identified in the sample.

13. The method of claim 10, wherein all 4 polymorphisms are identified in the sample.

14. A method for selecting a patient for a therapy to treat COVID comprising screening a biological sample isolated from the patient for 1 or more polymorphisms selected from the group of Table 1, and selecting a therapy to treat a patient at risk for progressing to severe COVID if 1 or more polymorphisms are identified in the sample.

15. The method of claim 14, wherein 2 or more polymorphisms are identified in the sample.

16. The method of claim 14, wherein 3 or more polymorphisms are identified in the sample.

17. The method of claim 14, wherein all 4 polymorphisms are identified in the sample.

18. The method of claim 14, wherein the therapy to treat a patient at risk for developing severe COVID is selected from a vaccine, a vaccine booster, corticosteroid, Paxlovid, Remdesivir, or Molnupiravir, or other equivalent therapy recommended for patients who are at high risk for progressing to severe COVID.

19. The method of claim 1, wherein the patient is a human patient.

20. The method of claim 1, wherein screening comprises contacting the biological sample or nucleic acid isolated from the biological sample with a labeled nucleic acid probe that specifically binds to a polymorphic region of interest having the polymorphic region, optionally a probe/primer pair as shown in Appendix A.

21. The method of claim 1, wherein the sample is at least one of blood, plasma, an original sample recently isolated from the patient, a fixed tissue, a frozen tissue, a biopsy tissue, a resection tissue, a microdissected tissue, or combinations thereof.

22. The method of claim 1, wherein the screening is by a method comprising PCR, PCR-RFLP, whole genome sequencing, sequencing, or microarray.

23. A probe or primer pair as shown in Appendix A and a preservative and/or a stabilizer for the probes and/or primer pairs.

24. A kit for performing the method of claim 1, comprising reagents to identify or determine the genotype of the sample and instructions for use.

25. A kit for performing the method of claim 14, comprising reagents to identify or determine the genotype of the sample and instructions for use.
